(19) United States
(12) Patent Application Publication
(10) Pub. No.: US 2024/0065793 A1
(43) Pub. Date: Feb. 29, 2024

(54) SYSTEMS, DEVICES, AND METHODS FOR MEASURING THE QUANTITY OF LOST BLOOD DURING SURGERY

(71) Applicant: SURGIBOX INC., Cambridge, MA (US)

(72) Inventor: Mike Horia Mihail Teodorescu, Cambridge, MA (US)

(73) Assignee: SURGIBOX INC., Cambridge, MA (US)

(21) Appl. No.: 17/897,172

(22) Filed: Aug. 28, 2022

(51) Int. Cl. A61B 46/23; A61B 5/02

(52) U.S. Cl. CPC A61B 46/23; A61B 5/02042; A61B 2046/205; A61B 2046/236; A61B 2562/0247; A61B 2562/043

(57) ABSTRACT

A blood-management-system for collecting blood from a surgical-site of a patient and for measuring the patient's blood loss is disclosed. The blood management system includes a drape configured to be disposed on the patient. The drape further includes an adhesive layer forming a seal between the drape and a region surrounding the surgical site. The blood management-system further includes a drape-collector disposed on the drape, in the proximity of the surgical-site. The drape-collector is configured to collect blood spilled on the surgical-site-layer, to store blood, and to evaluate the quantity of blood collected in the drape-collector. The blood-management-system further includes blood-containers configured to receive fluids from the drape, to store the fluids, and to measure the fluids quantity. The blood-management-system further includes a computing system configured to receive information from the drape-collector and the blood-container and to evaluate the amount of blood lost by the patient.

### BACKGROUND OF THE INVENTION

### 1. Field of Invention

The present invention relates to a method and a system, implemented as a surgical drape, for continuously monitoring the quantity and composition of the blood and fluids ejected from the body of a patient in circumstances such as during surgery or from bleeding wounds.

### 2. Discussion of the Background

Losing blood in large quantities is a threat to life. The lost blood should be replaced as fast as possible. Replacing the lost blood requires a good estimation of the lost blood volume. If too little blood of the lost quantity is replaced, the threat to life may remain. At the opposite end, overcorrecting for blood loss by transfusing too large a quantity of replacement blood into a patient's system (beyond what was lost) may itself be a threat to life, see for example references 1 and 2 listed at the end of the detailed description section (Agnihotri et al., 2014, and Clifford et al., 2017). These problems have been recognized early in the literature, see for example reference 3 (Thornton, 1963) and reference 4 (Desmond, 1973), and have recurrently been addressed by others, see for example reference 5, (Lee et al., 2006), reference 6 (Johar and Smith, 2009), and reference 7 (Alli and Hare, 2021).

Patent Application US20030060831A1 (Bonutti, 2003) discloses systems and methods for handling blood released during surgeries via a drape with a drain. However, the systems and methods in Bonutti have many shortcomings and do not provide for ways to determine the volume of the blood and fluids released during the surgery.

The measurements of accumulated blood volumes provide operators (e.g., surgeons, nurses, first-responders) with information regarding patient's total blood loss and the evolution in time of the blood loss. Such information is often life-saving information enabling medical professionals and first responders to provide appropriate medical care.

The problem of estimating the quantity of lost blood during surgeries remains essentially unsolved. The deficiencies of the current solutions relate to the poor accuracy of the employed blood loss measurements which leads to very large uncertainty in the measurements. It is important to note that in most circumstances using fixed shape blood collectors (e.g., containers with hard walls such as buckets) is not feasible. Specifically, the use of fixed shape blood containers is not feasible because of the variable dimensions and shapes of the patients' bodies and because of the various circumstances occurring during surgeries. Using fixed shape blood collectors is likely to result in faulty fluid collection and large measurement errors for the lost blood volume. The use of aspiration devices, as for example described in reference 9 (U.S. Pat. No. 11,160,602 by Shelton et al., 2021) is imprecise because corporeal fluids can be ejected far from the surgery place. Moreover, the fluids generated during the surgery and collected in the fluids collectors include not only blood but also other fluids (e.g., draining wounds, lymph fluid, amniotic fluid, other bodily fluids, etc.). Thus, in order to determine the quantity of the blood lost during the surgery it is also necessary to determine the composition of the collected fluids (i.e. the percentage of blood in the collected fluid, the quantities of other fluids mixed with the blood). Not taking into account the composition of the fluid may result in incorrect measurement of the blood loss and in substantial over-estimation of blood loss.

Various surgical drape configurations have been proposed. Some surgical drapes are configured to collect the blood or fluids generated at the surgical site. Essentially, these solutions collect fluids from large surface drapes and direct the fluids toward large fluid containers external to the drape. Some drapes may include a drainage hole that leads to an external container. Examples of such configurations are presented in reference 8 (US Patent Application US20030060831A1, by Bonutti, 2003), reference 10 (U.S. Pat. No. 5,167,613, by Karami and Vitaris, 1992), reference 11 (U.S. Pat. No. 5,465,735 by Patel, 1995), and reference 12 (U.S. Pat. No. 6,077,526 by Scully and McCabe, 2000). While some of these drapes can do partial collection of bodily fluids, no one is able to accurately collect and measure the bodily fluid quantity due to a number of technical challenges. Indeed, before the surgery, the quantity of ejected bodily fluids is unknown, moreover the distance from the surgery site to the place where the fluid will fall is unknown. When small quantities of fluids are released at small distances, for example less than half a liter (0.5 cubic dm) close to the surgery site, part of the fluid will wet and remain on the collecting drape, possibly dry, introducing significant measurement errors. In addition, for small fluid quantities, the use of large fluid containers inherently introduces larger measurement errors. What is needed for an accurate measurement is a drape able to collect, retain and measure fluids spilled both locally and distantly, with good accuracy.

There are known surgery drapes that are able to collect bodily fluids dropped close to the surgery place, with the collection performed for hygienical purposes. These drapes have no means to determine the quantity of the fluid collected, moreover cannot collect large quantities of fluids. There are known drapes that collect in canisters fluids spilled over a larger surface around the surgery place with the purpose of determining the fluid quantity. However, these drapes have rudimentary systems for measuring the fluid quantities, are inconvenient and provide low measurement accuracy. The drape systems disclosed herein do not have the above shortcomings and drawbacks.

The devices and methods disclosed by this application provide solutions to the above-described problem of collecting and determining of blood volume lost during surgery or in other circumstances. The invented systems and devices can be effectively used in time. The problem is solved via methods combining devices, sensors, statistical methods, and machine learning procedures.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

### SUMMARY OF THE INVENTION

Exemplary embodiments of the present invention provide blood-management-systems configured to collect blood generated during surgeries and other medical procedures performed on a patient and to evaluate the quantity of blood lost by the patient. Exemplary embodiments of the present invention also provide methods for collecting blood generated during surgeries and other medical procedures and for evaluating the quantity of blood lost by the patient.

Exemplary embodiments of the inventions may implement, among others, several innovations, namely (i) surgical drapes able to assume shapes that allow the secure collection of bodily fluids ejected from a surgical place or a wound, the said drapes embedding or having attached blood-containers for the collection of fluids; (ii) methods and devices for determining the volume of the collected bodily fluids accumulated in the blood-containers; (iii) methods and systems for estimating the fluid dried over the drape or remaining over the drape; (iv) methods and systems for estimating the blood flow from the body and for controlling the blood replenishment of the patient's body.

Innovative collecting systems and devices are disclosed. The collecting systems may include innovative shapes and innovative collecting surfaces consisting essentially of an innovative surgical drape having a specific shape that ensures the blood collection from the patient under surgery. The collecting devices may use a collecting drape with an internal pocket or pouch that may absorb bodily fluids. The systems may measure the weight or volume of the collected fluids by employing arrays of sensors included in the drape and an internal or external measurement and computation circuitry. The system may use a drape with restraining strings configured to enable the formation of draining surfaces. The system may use, in an embodiment, an inflatable frame and/or inflatable ribs that extend the drape boundaries to take the shape of collecting surfaces.

Innovative fluid and blood collecting containers are disclosed. The fluid and blood collecting containers are configured to work in conjunction with the fluid and blood collecting surface of a drape. The blood-containers may be controlled and maintained into certain shapes via rigid ribs, semi-rigid ribs, and/or inflatable ribs. The systems are designed so that the drape may be easily folded and packed into a minimal volume, while not compromising the usability and reliability of the drape and blood-containers. Innovative electrodes systems are disclosed.

Innovative methods for determining the quantity of the blood in the collecting blood containers is disclosed. The methods may be based on one or a combination of the following: impedance tomography, optical 3D measurements, resistance measurements, and impedance metric, colorimetric, spectroscopic, or chemical determination of the composition of the fluid in the blood-containers.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a diagram of an exemplary embodiment of a blood-management-system configured to collect blood from a patient during a surgical procedure.

FIG. 2(a) shows a top view of a blood-management-system including a drape and a blood-drape-collector configured to collect blood generated close to the surgical site.

FIG. 2(b) shows a cross-section through the drape-collector in FIG. 2(a).

FIG. 3 shows an exemplary embodiment of a blood-management-system including a plurality of blood-drape-collectors disposed around the surgical site.

FIG. 4 shows an exemplary embodiment of a signal-reading-system connected with a plurality of sensors.

FIG. 5 shows an exemplary embodiment of a signal-processing-system configured to receive and process signals from the sensors of the blood-drape-collector.

FIG. 6A shows an exemplary embodiment of a blood-management-system where the blood-containers are disposed along the sides of the drape and the blood-containers further include a fluid-measurement-system configured to measure the volume and the composition of the fluids in the blood containers.

FIG. 6B shows a detailed enlarged view of the fluid-measurement-system in FIG. 6A.

FIG. 7 shows an exemplary embodiment of a resistivity-measuring-system configured to measure the resistivity of the fluids in a blood-container.

FIG. 8 shows an exemplary embodiment of a blood-management-system including a drape which may have a saddle-shape, fluid-containers, and a set of gutters.

FIG. 9 shows an exemplary embodiment of a blood-management-system including a drape which may have a saddle shape, fluid-containers, a set of parabolic supports, and a set of linear supports.

FIG. 10 shows an exemplary embodiment of a blood-container including one or more ribs configured to reinforce the walls of the blood-container.

FIG. 11 shows an exemplary embodiment of the blood-management-system including an inflatable enclosure.

### DETAILED DESCRIPTION

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity.

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

It will be understood that when an element or layer is referred to as being “on” or “connected to” another element or layer, it can be directly on or directly connected to the other element or layer, or intervening elements or layers may be present. In contrast, when an element or layer is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XY, YY, YZ, ZZ).

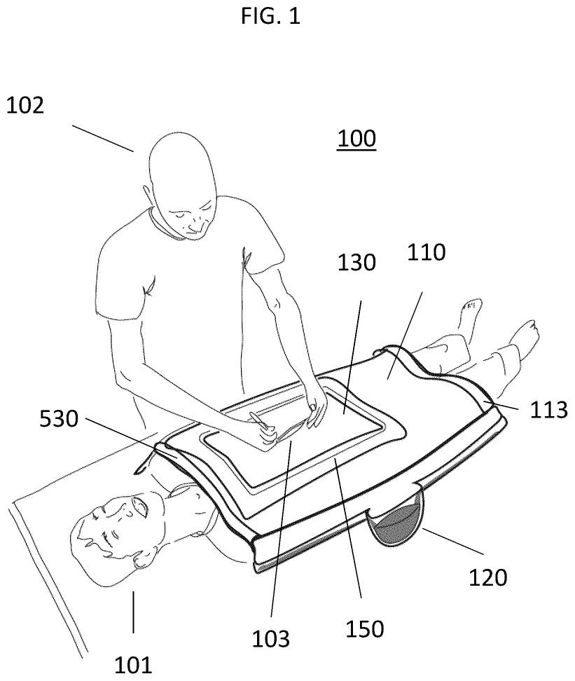

FIG. 1 shows a diagram of an exemplary embodiment of a blood collection and management system 100 (hereinafter referred as “blood-management-system”). The blood-management-system 100 is configured to be disposed on a patient 101 (as shown in FIG. 1) while a surgical procedure is performed on the patient by one or more operators 102; to collect blood and other fluids generated during the surgical procedure; and to evaluate the amount of blood lost by the patient at various times during the surgery. The blood-management-system may be further configured to determine periodically/pseudo-continuously (e.g., every 5 seconds) the amount of blood lost by the patient. The blood-management-system may be further configured to provide information to the operators 102 about the measured blood loss.

The blood-management-system 100 may include a drape system 110, one or more drape-blood-collector systems 150, one or more blood-container systems 120, and a control and computing system. The drape system 100 is configured to collected blood and other fluids generated (e.g., coming from the surgical site 103 on the patient's body) during the surgery and to direct the blood and fluids to the blood-container system 120. The drape-blood-collector systems are configured to collect and measure blood spilling on the drape region close to the surgical site 103. The blood-container systems 120 are configured to collect and measure blood spilling away from the surgical site 103. The control and computing system may be configured to receive data from sensors of the systems, to process the data, and to control various processes.

The drape system 110 may be referred hereinafter as “drape”, a drape-blood-collector system 150 may be referred hereinafter as “drape-collector”; a blood-container-system 120 may be referred hereinafter as “blood-container”; and the control and computing system may be referred hereinafter as “computer”.

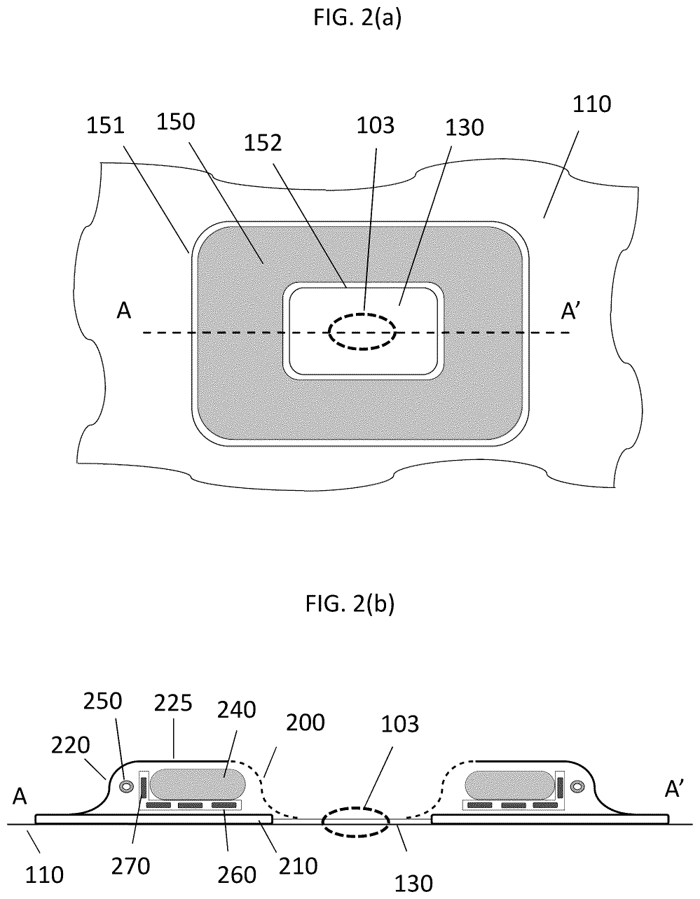

With reference to FIGS. 1 and 2, the blood collecting drape 110 is an essentially flexible, disposable drape of suitable shape and structure, made of one or several layers of suitable materials. The drape or at least its upper layer may be made of materials that are impermeable (impervious) to bodily fluids and that are repellant to (not wettable by) the bodily fluids, such that the bodily fluids are not retained on that part of the surface of the drape. The surface of the drape may be covered by a thin layer or may include anticoagulant substances, such as to minimize the quantity of blood remaining liquid or solid on the surface. Several layers composing the drape may be laminated.

The drape has one or several surgical-site-layers 130 covering the region where the surgery is performed (i.e., surgical-site 103). Part of the surgical-site-layers 130 are configured to be removed or cut-through prior to surgery so as to allow the surgeon to access the operating area/surgical-site. The surgical-site-layers 130 may include a perforated region which may be removed, thereby creating a window to the surgical site. The drape-blood-collector 150 (see FIG. 2(a), gray region 150) may include several material layers and may be connected at the edges with the surgical-site-layers 130 and the rest of the drape. The surgical-site-layers 130 and the layers of 150 may include medical grade adhesive patches able to tightly adhere to the body of the patient. The surgical-site-layers 130 and the layers of 150 may in some embodiments include antimicrobial surfaces.

In a non-limiting embodiment, the bottom layer of 150 includes a layer of adhesive; a second layer which may consist of an impermeable material covering the entire surface 110 of the drape, and one or several containers/recipients which may include sensors as in FIG. 2(b). In an alternative embodiment, the surgical-site-layers 130 (e.g., entire surgical window) may consist of a thin film with adhesive properties that can be cut through and no separate region 150 is necessary. In an alternative embodiment, surgical-site-layers 130 can be made of a non-adhesive material that is secured to the patient through straps, or gravity, or some other means, and may have perforations allowing for easy removal of the material in order to expose a surgical site. In many implementations, with or without specific adhesive patches or separate windows, it is essential that the boundaries of the surgery region or regions tightly adhere to the body of the patient such that fluids do not penetrate under the part of the drape outside the surgical-site. The adhesive surgery patch or the adhesive borders are hermetically connected to the remaining surface of the disclosed drapes and represent an integral part of the drapes.

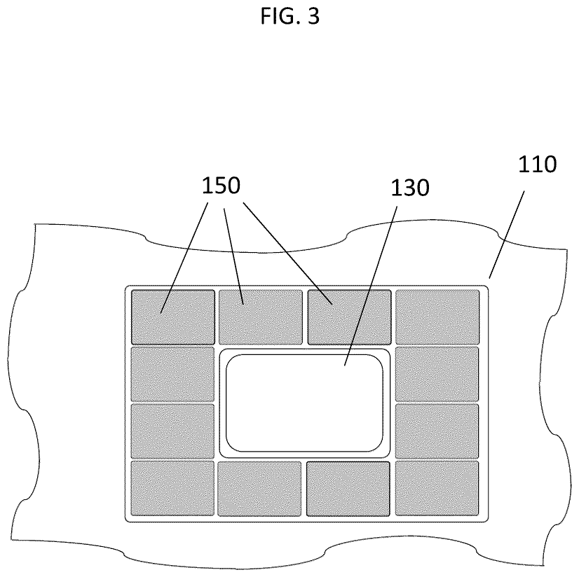

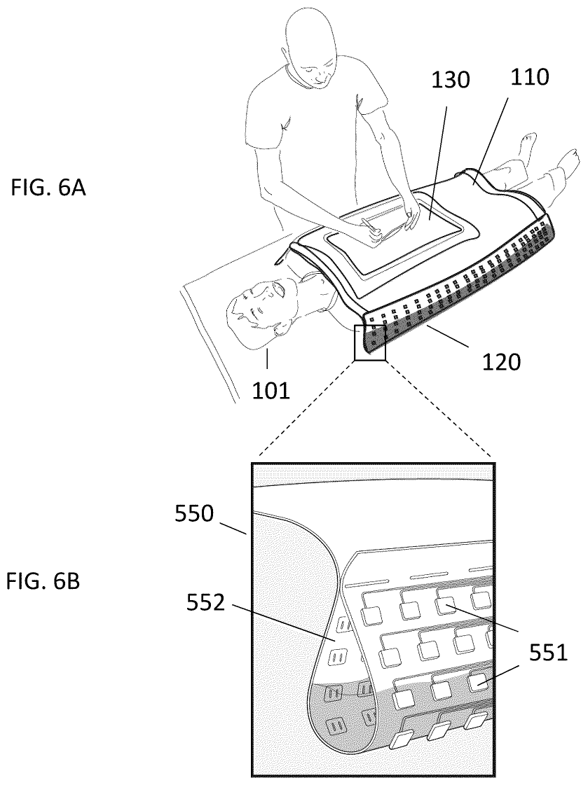

The surface of the drape 110 allows the flow of fluids to be directed to particular locations on the drape which may contain collectors, sensors, ports for drainage, or a combination thereof. FIGS. 2(a) and 3 show various configurations of drape surfaces. FIG. 1 shows a drape with channels and/or borders 113 and 530 that direct fluid away from the surgical-site 103 with an attachment border such as borders 113 (which may, in a non-limiting embodiment be an adhesive border) into blood-containers 120. Sensors may be incorporated into the blood-containers 120, forming structures inside the container which can be seen in the embodiment shown in FIGS. 6A and 6B. Sensors are also included in various other embodiments.

The flexible collecting drape 110 may be configured to be disposed on top of the patient so as to surround one or more operating areas of the patient where blood is generated and to collect the generated blood and fluids causing them to flow in specified directions (e.g., toward the blood-containers 120 at a distance from the surgery site(s)) or causing the fluid to accumulate in the blood-containers 120. The blood-containers 120 may be embedded in the drape 110 around the surgery site(s). For embodiments incorporated in the drape, the drape-collectors may surround one or several surgical-sites 103, while the borders of the collecting surface around the operating areas tightly attach/seal to the body of the patient and do not allow blood flow under the surface of the collecting drape 110. In addition, a drape-blood-collector 150 may be provided around the surgical-site.

The flexible collecting drape 110 may include one or more layers made of impermeable or permeable materials suitable for a surgical drape, such as rubber, plastic films, thermoplastics or a combination thereof that may include fabric, plastic, latex, thermoplastics, polyurethane, polycarbonate, acetal copolymer polyoxymethlene, polyethylene, acetal homopolymer polyoxymethylene, nitrile, vinyl, polyisoprene, neoprene, acrylic, nylon, polypropylene, polystyrene, nonwoven fabrics, spunbond fabrics, extruded materials or other thermoplastics or combinations thereof. In some embodiments, combinations of materials such as films with fabrics may be preferred, if the materials properties are sufficiently elastic, impermeable, comfortable, and/or non-brittle to act as a cloth-like material.

Many current surgical drapes have a specified area represented by a sterile patch that adheres to the skin of the patient. In a preferred embodiment, the drape includes an area having a sterile patch that adheres to the skin of the patient, wherein the contact between the patch and the skin is configured to prevent bodily fluids from penetrating under the drape.

In many surgeries, the quantity of lost blood is moderate, less than 1 cubic decimeter (1 liter), and the blood is not ejected at large distance, spilling over a small distance around the surgery place. In such cases, the collection of the blood and possibly other corporeal fluids may be performed locally, around the surgical site, with the fluids collected into one or several drape-blood-collectors 150 embedded in a special drape, as described hereinafter.

The drape-collector 150 described hereinafter is a system for measuring blood loss under circumstances such as the ones described in the paragraph above. The drape-collector 150 may be further configured to collect the blood generated by a bleeding wound of a patient. The drape-collector 150 is configured to collect low quantities of blood spilled close to the surgical site whereas the blood-containers 120 are configured to collect large quantities of blood which are directed towards the edges of the drapes.

The drape-collector 150 for receiving bodily fluids generated during surgery is placed close and around to the surgical-site 103 and/or near or at the edge of the surgical-site. FIG. 2(a) shows the drape-collector 150 from the above (direction perpendicular on the drape) whereas FIG. 2(b) shows a cross-section through drape-collector 150 (see cross-section A-A′ in FIG. 2(a)). The drape-collector 150 may include one or more permeable-sheets 200 which may be permeable to bodily fluids. The drape-collector 150 may include or may be attached to an impermeable-lower-sheet 210. The permeable-sheets 200 may be joined with the layer 130 (which may be a surgical patch) and/or may be joined with the impermeable-lower-sheet 210. The permeable-sheet 200 may be a perforated sheet, or a sheet with the structure of a thin mesh (e.g., structure similar to a sieve). The impermeable-lower-sheet 210 may be thick enough to avoid wrinkles, but elastic enough to adapt to the shape of the body of the patient. The drape-collector 150 may include or may be attached to an impermeable-upper-sheet 220.

The permeable-sheets 200 may be joined over some of the edges with an impermeable-sheet 220. The impermeable-sheets 220 are disposed on an outer-edge 151 of the drape-collectors 150 (as seen in FIGS. 2(a) and 2(b)). The permeable-sheets 200 are disposed on the surgical-site-edge 152 of the drape-collector 150 (as seen in FIGS. 2(a) and 2(b)).

The sheets 200, 210 and 220 form a fluids-enclosure 225 (e.g., a pouch) allowing fluids to enter the fluids-enclosure 225 through the permeable-sheets 200 at the surgical-site-edges 152 bordering the surgical patch. In FIG. 2(b), A and A′ show the two sides of the drape at the intersection of the cross-section in FIG. 2(a).

The drape-collector 150 may further include fluid-retention-elements 240 which may be disposed inside the fluids-enclosure 225 formed by sheets 200, 210 and 220. The fluid-retention-elements 240 are configured to store the fluid in the enclosure. The fluid-retention-elements 240 may be made of a fluid absorbing and retention material that has low mass in a large volume and large capacity of absorbing and retaining fluids (may be a sponge or sponge-like absorbent material, in a non-limiting embodiment). The fluid-retention-elements 240 may have an internal cellular structure. The cells of the cellular structure may have a height essentially equal with that of the fluid-retention-elements and may be opened upside, allowing the fluids to penetrate the cells. The fluid-retention-elements may be filled with an absorbent material or a combination of absorbent materials.

The absorbent material may be any of the known non-toxic and non-fume generating materials having one or more of the following properties: are stable enough for being sterilized, have high retention levels, and/or are recyclable. The material may be a hydrophilic material which absorbs blood. Examples of such materials are air laid composites, non-woven fabrics made of natural or synthetic fibers, powders such as sodium polyacrylate or potassium polyacrylate, which can absorb several hundred times their mass in water. Several sources disclose the composition and structure of such materials, including U.S. Pat. Nos. 5,167,613, 5,465,735, 6,077,526. The highly absorbent materials can include combinations of the above and combinations with nano-absorbents that have also a role of bacteria and virus absorption (Ojha, 2020), thus contributing to the hygiene at the surgery place.

The drape-collector 150 may further include the end or ends of one or several tubes 250 configured to drain the fluids from inside the drape collector, such as fluids which are not absorbed by the fluid-retention-elements 240. The other end of the tubes 250 may direct the drain fluids into one or more external containers, external to the drape or attached to the drape toward its distant edges.

The drape-collector 150 may further include bottom-sensors 260 and side-sensors 270 for measuring the quantity of the fluid accumulated in the drape-collector 150. The bottom-sensors 260 may be pressure sensors. The bottom-sensors 260 may be disposed under the fluid-retention-elements 240 (as seen in FIG. 2(b)) and may be configured to measure the quantity of fluids in the fluid-retention-elements 240 by measuring the pressure change caused by the weight of the fluids accumulated in the fluid-retention-elements 240. The pressure bottom-sensors 260 may be disposed in an array and may form a sensor array.

The side-sensors 270 may be pressure sensors. The side-sensors 270 may be disposed on the edges of the fluids-enclosure 225 (as seen in FIG. 2(b)) and may be configured to measure pressure changes caused by the fluids accumulated in the fluids-enclosure 225. The measurements performed by the side-sensors 270 may be used for correcting for the non-horizontality of the fluids-enclosure 225 and the drape-collector 150. Side-sensors 270 may be disposed in a position perpendicular to the drape so as to measure pressure parallel to the drape 110 and at the non-permeable edges of the drape-collector 150 (see FIG. 2(b)).

The drape-collector 150 may further include one or more of the following sensors: sensors for pH, biochemical sensors for chemical composition, sensor for resistivity, colorimetric sensors, optical or IR spectroscopy sensors, ultrasound velocity sensors, and sensors for electrical impedance spectroscopy.

The total weight of the fluid accumulated by the fluids-enclosure 225 (and the drape-collector 150) may by determined by measuring the pressures on the array of sensors 260 and 270 at an initial-time (before any liquid has been accumulated into the fluids-enclosure 225) and at a certain measurement-time (after liquid has been accumulated into the fluids-enclosure 225). A total pressure/weight at a certain measurement-time may be evaluated by integrating, over the area of the sensor arrays, the pressure readings of the individual sensors. Various types of pressure sensors may be used, such as: piezoresistive sensors, piezoelectric sensors, and/or capacitive pressure sensors (see for example references 13-15, Mannsfeld et al., 2010; Kim, Yang, and Oh, 2021; and Xiong et al., 2020). The pressure sensors are selected such that they provide the required sensitivity for measuring at least the normal pressure and preferably sensitive to both normal and tangential pressure, for compensation of the local non-horizontality of the drape.

The sensors 260 and 270 may be implemented via flexible electronics and thin films technology so that the sensor arrays are essentially flexible. The sensors may include thin films of metals, metal oxides including indium oxide, indium tin oxide (see reference 16, Bouden et al., 2016), and graphene (see reference 17 by Luo et al., 2018). The sensors are insulated in a thin sheet blister or are protected by nonpermeable and dielectric thin sheets such that the sensors are electrically isolated from the fluids.

FIG. 3 shows an exemplary embodiment of a blood-management-system including a plurality of blood-drape-collectors 150 disposed around the surgical site. Unlike the blood-management-system in FIG. 2(a) which includes one contiguous blood-collector 150, the blood-management-system of FIG. 3 includes a plurality of separate blood-collectors 150 (e.g. see the 12 blood-collectors in FIG. 3).

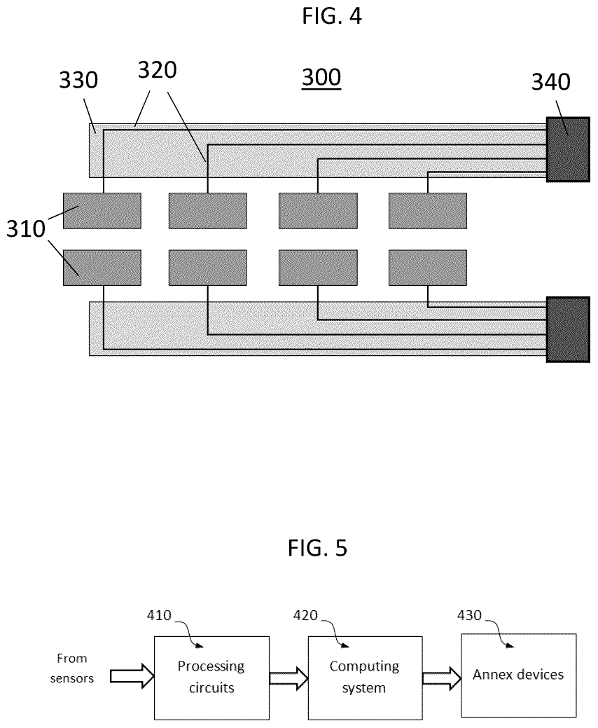

FIG. 4 shows an exemplary embodiments of a signal-reading-system 300 which may be used to acquire signals from sensors and electrodes and to control the electrical input/output of the sensors and electrodes. The signal-reading-system 300 may include or may be connected with sensors 260 and 270. The sensors 260 and 270 may be disposed in arrays. The sensors 260 and 270 may be resistive and/or electrical impedance sensors. The sensors 260 and 270 may be configured to measure quantities other than pressure.

Some of the sensors may include electrodes 310 which may be included in the fluids enclosure 225 and which may contact the fluids accumulated in the fluids-enclosure 225. The thin flexible metallic connections 320 may be printed or otherwise manufactured on the base of the drape, which may be include sheet 210 (see FIG. 2(b)). The electrical connections are covered by an insulating sheet 330 for avoiding the electrical conduction through blood. Typically, one measures currents produced by specified voltages between paired, adjacent electrodes, or one measures voltages produced by specify currents, or one measures voltages when between neighboring electrodes a specified current is established, with measurements performed at one or several specified frequencies, or in a specified frequency range of the applied voltages or currents. The measurements inform on the resistance or impedance between electrodes. The resistances or impedances are then converted in quantity of conductive fluid joining the respective electrodes.

Those skilled in the art will recognize that these measurement methods are well known and that only their use with the specific configuration of the disclosed drapes is novel. Also, those skilled in the art will recognize that the impedance measurement method for the volume of fluid includes a capacitive measurement method, where mostly the losses of the capacitances depend on the fluid content. For this method, the errors tend however to be higher; in addition, for this method the electrodes have to be isolated from the fluid. In FIG. 4, connectors 340 may be used to connect the sensors (e.g., sensors 260 and 270) or the electrodes 310 to the measurement circuitry.

FIG. 5 shows an exemplary embodiment of a signal-processing-system configured to receive and process signals from the sensors or the electrodes of the blood-management-system. As seen in FIG. 5, the signals from the array(s) of sensors are fed to signal processing circuits 410, via the signal-reading-system 300, and the output from them is passed to a computing system 420 for the determination of the blood volume lost. Based on the blood volume result, the computing system 420 generates control signals for annex devices 430, such as the blood delivering device and alarms, and may store the dynamics of the process and may send information to other systems.

For reducing the uncertainties of the measurement, the portion of the drape sheet carrying the sensors, while elastic enough for it to conform to the shape of the patient body (and should be flexible to allow for a wide variety of patient body shapes), should be essentially non-extendable, for the dimensions of the electrodes and the distances between them along the surface of the drape remains same. If the portion of the drape that contains the sensors is essentially not extendable, the computation of the volume of the fluids is affected by smaller uncertainties and thus is less complex than the computation for the entire body impedance tomography, because the boundary where the electrodes are positioned (lower side) is partly known, moreover the thickness of the conductive layer is small and the medium is almost homogeneous.

In FIGS. 6A and 6B, a non-limiting embodiment shows a sensor portion of the drape to be disposed outside the patient body or any drape attachment points to the patient (including adhesive portions), and thus not needing to conform to various patient body habitus, as the fluid reservoirs and sensors are outside the surgical site. Those skilled in the art will recognize similarities in the problem of fluid volume computation and the human body tomography, as well as in the computation method.

Although less frequent, there are surgery cases when blood mixes with other fluid, e.g., purulent fluids, lymph fluid, amniotic fluid, other bodily fluids, etc. For these cases, information on the composition of the collected fluid is required for the correct estimation of the lost blood volume. Moreover, blood properties may vary even when no other fluids are mixed with the blood. The resistivity and impedance at low frequencies of the blood varies largely with the blood composition (see references by Mohapatra and Hill, 1975; Hirsch et al. 1950; Schwan and Kay, 1956).

The blood-management-system may be configured to perform a wide variety of measurements on the blood accumulated into the blood-containers and drape-blood-collectors. A larger number of properties measured by the blood-management-system will enable the system to more accurately determine/calculate the blood volume and to reduce uncertainties related to the electrical properties of the fluid. The blood-management-system may be configured to determine the resistivity and impedance at a set of specified frequencies. The blood-management-system may be further configured to determine other properties of the fluid, such as temperature, pH (Kellum, 2000), and colorimetric properties (Szolga and Mudure, 2020). This broader set of measurements enables the blood-management-system to better infer the relation between resistivity and impedance of the fluid at specified frequencies and the blood concentration in the fluid.

The blood-management-system is configured to perform measurements on the fluids accumulated into the blood-containers so as to enable the determination of the fluids composition (e.g., the blood-management-system may determine that 80% of the fluids in the blood-containers are blood). The blood-management-system is configured to perform measurements on the fluids so as to enable the determination of the composition of the blood.

The blood-management-system may further include one or more of the following sensors for improving the determination of the fluid composition: biochemical sensors, sensors for optical or IR spectroscopy, and sensors for ultrasound-based measurement of the sound velocity in the fluid. At least one of the above sensors may be included in each of the blood-containers 150. The signals from these sensors are fed to processing circuits 410 and then to the computing system 420, which corrects the computed blood volume.

The blood-management-system may further include a compressibility measuring device configured to determine if the fluid includes significant amounts of gases. The compressibility measuring device may be similar to the one described in U.S. Pat. No. 10,088,454.

Drapes and related measuring system for measuring blood loss in large quantities and/or scattered over a large area of the drape are described hereinafter with reference to the drawings. In major surgeries, large quantities of blood may be generated and, as a result, the blood cannot be stored in relatively small fluid-containers (such as drape-fluid-collectors 150) embedded into a surgical drape. Moreover, blood may be ejected at relatively large distances from the surgery site. While for transport the drape should be foldable into a reasonably small package, during use it should maintain the shape assigned for fluid collection. This can be achieved in several ways.

Essential for the operation of the flexible collecting surface and the entire system disclosed is that the collecting drape 110 preserves, during fluid collection, a shape such that it drains the bodily fluids toward the blood-containers 120 attached to it, where the fluids accumulate. There are several solutions that embody this requirement, several of them described subsequently without restraining the coverage of the disclosure only to these solutions. Shapes of the collecting drape 110 for fluid collection are all characterized by parts of the drape, typically toward the shoulders and the legs of the patient, being higher than the other parts of the drape. In addition, at least one part of the drape lowers toward collecting-gutters or directly toward one or several blood-containers 120. The blood-containers may be formed as an integral part of the drape; may be attached or connected to the drape; or may be detached yet placed in a suitable position with respect to the drape.

A drape for fluid collection on large surfaces is essentially a drape with three-dimensional (3D) shape configured to collect the blood generated from one or more operating areas of a patient on which surgery is performed. The third dimension of the drape may be much smaller than the other two dimensions, such that the non-planarity of the drape does not hamper the surgeons' activity. The actual shape and components of the drape depends on the implementation. Several embodiments/implementations are disclosed hereinafter. All the implementations of drapes with non-planar shapes feature one or several blood-containers 120 for fluid collection.

FIG. 1 shows an exemplary embodiment of the blood-management-system including a drape with large collecting surface. The blood-management-system includes a blood collecting drape 110 and one or more blood-containers 120 annexed to the drape or being part of the drape. The blood-containers 120 may be disposed on the lateral, longer edges of the drape.

The blood collecting drape 110 is connected with or may incorporate at one or more edges the one or more blood-containers 120. The collecting drape is provided with or has its lateral edges configured such that the fluid collected by the collecting drape 110 is directed to the blood-containers 120. The drape 110 may also include one or more elevated-edges 530 configured to direct the fluid flow toward the lateral containers 120. The elevated-edges 530 may be thicker and/or semi-rigid so as to keep their shape and to provide a barrier for the fluids flow (e.g. blood, bodily fluids). The drape may also include (or be attached to) around the surgical-site 103 one or more local drape-blood-collectors 150.

FIGS. 1, and 6-10 show several implementations/exemplary embodiments of the blood-containers 120. Blood-containers 120 are configured to collect all or part of the blood and fluids spilled on the drape 110. In most situations, blood-containers 120 are configured to collect and store large quantities of blood and fluids, whereas the drape-collectors 150 are configured to collect smaller quantities of blood and fluids.

FIG. 6A shows an exemplary embodiment where the blood-containers 120 are disposed along the sides of the drape 110 (e.g., both on the left and right side of the patient). The blood-container may be formed essentially as a fold in the edge of drape 110 (as seen in FIGS. 6A and-6B). The blood-containers are disposed at a lower elevation than other parts of the drape. The blood and fluids spilled on the drape 110 are directed (e.g., by gravity) to the blood-containers 120, enter the blood-container 120 through gaps in between the folds, and are accumulated/stored into the blood-containers 120. The walls of the blood-container are made of flexible material thereby allowing for changes in shape of the container so that the blood-containers adjust/adapt their shape to the shape of the patient body and its surroundings.

FIGS. 8 and 9 show another exemplary embodiment of the blood-containers 120 wherein the blood-containers have a side-pocket shape. The side-pocket shape may be essentially a quarter ovoid shape. The blood-containers are disposed at a lower elevation than other parts of the drape. The blood and fluids spilled on the drape 110 are directed (e.g., by gravity) and accumulated/stored into the blood-containers 120.

The blood-containers 120 may be made from the same material as the flexible impermeable layer of the drape and may be a continuation of that layer. The walls of the blood collection blood-containers 120 may be flexible and their shape may adapt to the circumstances of the surgery. The blood-containers 120 may have an essentially concave shape and may maintain their concavity while allowing for the deformations of the walls. Since the blood collection blood-containers 120 are made of flexible material layers, they will adapt to the shape of objects they touch/contact (e.g., table, other equipment, the patient, surgeon). Thus, their shape will be largely uncontrolled and undetermined during the operation (dependent on the geometrical configurations and circumstances of the surroundings).

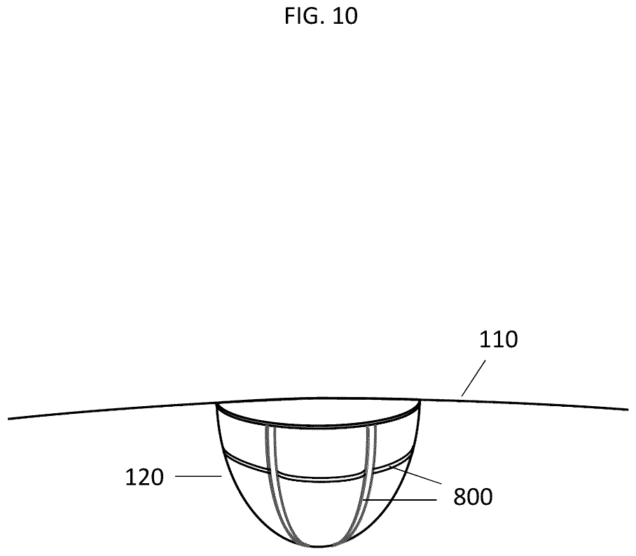

FIG. 10 shows an exemplary embodiment of a blood-container 120 which may include one or more ribs 800 configured to reinforce the walls of the blood-container. The ribs may be rigid ribs and/or may be semi-rigid ribs which allow for some shape deformation (e.g., made for example of thin plastic slabs) but not for very large deformations. The semi-rigid ribs are configured to provide a structure and support to the blood-containers while allowing some flexibility. The ribs 800 may be disposed both longitudinally and transversally on the blood-containers. The ribs 800 are configured to maintain the shape of the blood-containers 120 within a certain predetermined range of shapes and to prevent too large deformations of the blood-containers. The ribs may be made of semi-rigid plastic which may be bendable and elastically or plastically deformable. The ribs may also be made of semi-rigid wires of various shapes or may be inflatable ribs. The blood-containers 120 may also have a thicker collar, as a transversal rib.

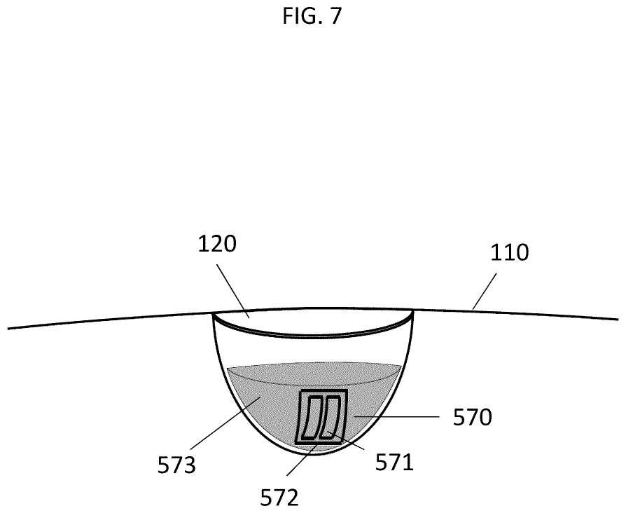

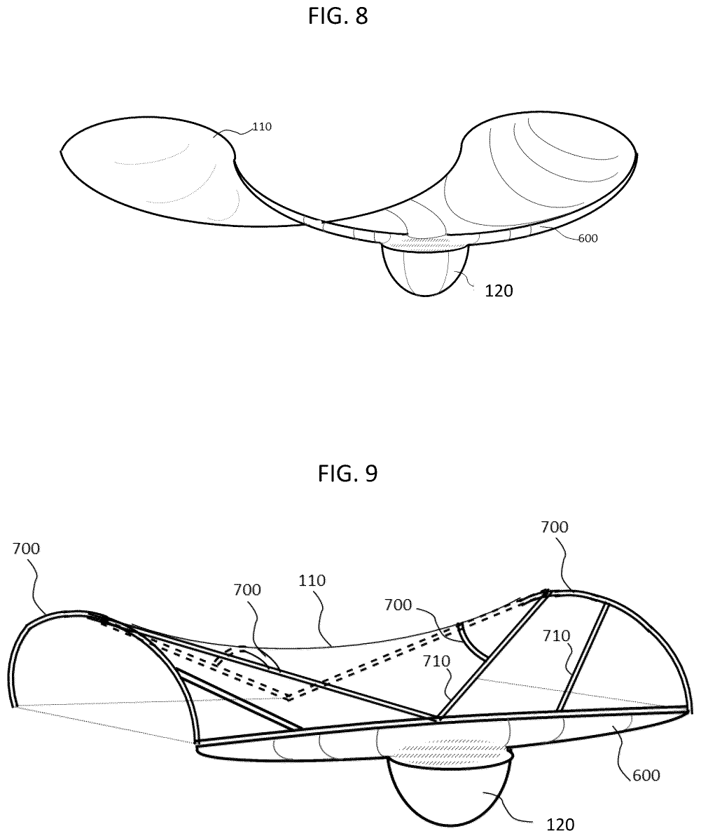

FIG. 6A shows an exemplary embodiment of a blood-management-system wherein the blood-container 120 further includes a fluid-measurement-system 550. The fluid-measurement-system 550 is configured to determine one or more of: the volume, the weight, the composition of the fluids accumulated in the blood-container 120, the quantity of blood in the blood-container, information about the composition of the blood. The fluid-measurement-system described herein may be incorporated and may operate with other types of blood-containers 120, such as the ones shown and described with reference to FIGS. 7-10.

A simple way of determining the weight of the fluids in a container is to measure the weight of the container with fluid and subtract the container weight when empty. For weighting the containers with fluids, one may use stress gauges with suitable circuitry, with the stress gauges placed in one or several places on the joint between the container and the drape, either the joint is simply a part of a sheet of the drape or otherwise made. Other types of sensors can be used for the same purpose. The method described in this paragraph, essentially representing a weighting method, has the drawback that the positions of the drape and of the containers make the measurement imprecise.

In another implementation, where the blood collecting containers hang by a strip from the drape, where the strip elongates under the weight, a displacement transducer or a sheet thickness gauge can be used to indirectly determine the weight. Those skilled in the art will recognize that, in this case, there are several other possible, known solutions for determining the weight.

FIG. 6B shows a detailed enlarged view of the fluid-measurement-system 550 wherein the fluid-measurement-system is configured to determine the volume of the fluids inside blood-container 120. Determining the volume of the fluid in blood-containers with flexible walls, such as in FIG. 6A, poses difficulties. The shape of the blood-containers 120 may change when the patient and/or the surgeons move, for different use circumstances, from patient to patient.

The fluid-measurement-system 550 may include an array of electrodes 551 in contact with the fluids. The electrodes are disposed on the interior walls 552 of the blood-container 120. The fluid-measurement-system may include a control-circuit configured to supply voltages/currents to the electrodes and to acquire signals from the electrodes. The fluid measurement system is based on impedance tomography or impedance resistivity techniques and is configured to estimate the volume of fluids (e.g., blood, other bodily fluids) accumulated in the containers 120. The electrodes 551 of the fluid-measurement-system are configured to be used as in typical impedance tomography (e.g., in medicine) and as in electrical resistance tomography (e.g., in geology). For example, control-circuits may send measuring current signals to some of the electrodes and may receive/measure resulting potential values (voltages) between the other electrodes. The measured signals are used to evaluate the shape and volume occupied by the fluid in the blood-container via procedures/algorithms such as the ones used in tomography (known in the art). For example, the measured signals are sent to a computing system that solves the problem known as the ‘non-linear inverse problem with unknown boundary’ to determine essentially the shape and volume occupied by the fluid in the blood-container. Any of the known algorithms for solving the said problem of impedance tomography may be used. The solving of the problem is easier than that for the impedance tomography of the human body because the fluid is relatively homogeneous and the shape of the container varies in a known range. The fluid measurement system provides reliable ways to estimate the volume of fluids in flexible blood-containers of variable shapes, thereby eliminating the disadvantages of known methods for evaluating blood loss.

The problem of determining the blood loss volume from an unknown volume of bodily fluids with unknown mixture proportions can be framed in general terms as a volume determination problem for an essentially unknown mixture of fluids in a flexible blood-container of variable shape, wherein the mixture of fluids includes one known fluid with well-known properties (in this case, the blood). The variables of the problem may include the shape of the blood-container, the fluids mixed in the flexible collecting blood-containers, the ratios of the fluids in the mixture, the physical properties of the mixture, and the volume occupied by the fluids in the blood-containers. Accurate methods for determining the blood loss may include several different measurements, combining the results of these measurements, performing computer modeling/simulations, performing calculations/computations.

While the fluid-measurement-system 550 may employ impedance tomographic methods to determine the volume of fluids inside the blood-containers, more accurate results may be obtained by performing additional supplementary measurements. Impedance tomographic methods may be affected by the variable shape of the surface of the blood-containers, by the composition of the fluid, and by its temperature. The fluid-measurement-system 550 may be further configured to determine the density, temperature, and/or resistivity of the fluids in the container 120. The fluid-measurement-system 550 may provide the results of the measured densities, temperature, and resistivity to the computing system. The fluid-measurement-system 550 may use the measured densities, temperatures, and/or resistivities together with the tomographic measurements to calculate/evaluate both the volume and the composition of the mixture of fluids (e.g., blood, urine) in the container 120. The calculation/evaluation may be performed via procedures known in the art. The evaluated composition of the mixture of fluids may include information about the amount of blood included in the mixture. For example, the calculations may provide that blood constitutes 80% of the total mixture. The combination of measurements provides accurate blood-loss estimates even when the blood is mixed with other body fluids.

The fluid volume measurement from each of the blood-containers and the signals from the sensors for temperature, resistivity, fluid density and other sensors may be sent to a processor configured to combine all the measurements from all blood-containers and to provide an estimate for the entire amount of blood lost by the patient. The processor may control suitable displays and alarms, as well as blood transfusion pumps.

As mentioned, the fluids-measurement-system may use a plurality of electrodes 551 of small dimensions to determine the resistance and the impedance between the electrodes. The electrodes array 551 and/or the associated control-circuits may be fabricated and attached/incorporated in the walls 552 via known methods, such as the ones used in the field of flexible electronics. The plurality of electrodes 551 may come with connections configured to be switched between current generators and potential measuring devices in a specified manner controlled by the computing system. The electrodes 551 may be made of one or more metallic conductors or other conductive materials. The electrodes 551 may be fabricated and incorporated in or deposited on the walls 552 of the flexible blood-containers according to known technologies, such as flexible electronics technology (see Wong and Salleo, 2009). Electronic components of the fluid-measurement-system may be implemented as flexible electronics circuits.

The processor controls a multiplexing system for selecting, out of the electrodes 551, subsets of current injection electrodes and electrodes for the measurement of potential, wherein the subsets may consist of variable numbers of the electrodes. The processor is also configured to implement various measurement protocols, methods, and operation regimes.

The processor may switch between different measurement protocols and operation regimes, such as: measurements of electrical resistance at low frequencies; measurements of impedance at one or several high frequencies; measurements of current through the electrodes; measurements of voltage between two electrodes; etc. For example, the processor may implement a two electrodes measurement protocol, or a 4-probe measurement of resistance or impedance between electrodes. For most of the protocols and operation regimes, the processor is configured to receive data from the electrodes, to store data in memories, to perform data processing, and to output the results of processing, including volume of the fluids in a certain flexible blood-container 120, density of the fluids, estimated percentage of blood, and blood volume. Ultrasound measurements may be performed on the fluids in the container so as to determine the velocity of ultrasounds in the fluid. The density of the fluids in the blood-container may be determined indirectly from the velocity of the ultrasound via formulas or procedures known to the skilled artisan.

FIG. 7 shows an exemplary embodiment of a resistivity-measuring-system 570 of the fluids in a blood-container 120. The resistivity-measuring-system 570 may include resistivity measuring electrodes 571. The resistivity-measuring-system 570 may be positioned close to the basis (bottom) of the blood-container 120 so that measurements can be performed even when the blood-container includes small amounts of collected fluid 573 and so as to minimize the effects that proximity to the surface of the fluid have on the measurements. This way, the accuracy of the measurements increases. Further, for reducing the influence of other nearby electrodes pertaining to the system for determining the volume of the fluid, the electrodes 571 for resistivity measurement may be shielded by the grounded-frame 572. The electrodes are connected to a suitable current or voltage generator, depending on the measurement method used, and to the suitable measurement circuits. The current or voltage representing the measured value is fed to an analog to digital converter which sends data to the processor. The resistivity measuring system is configured to perform repeat measurements (e.g., periodically) since the composition of the spilled fluid may change during surgery and thus the measurement system may need the resistivity parameter to periodically calculate the volume of the blood.

The resistivity-measuring-system 570 may use the two-electrodes method for determining the resistivity of the liquid, as shown in FIG. 7. The resistivity-measuring-system 570 may use four electrodes 571 configured to implement the four-probe method for determining resistivity, which is a more precise method. Those skilled in the field would understand that a combination of measurements, including resistivity at low frequencies and impedance at higher frequencies may be used so as to increase accuracy. In alternative embodiments, more than four probes can be used.

Several sets of electrodes 571 for measuring resistivity may be used. The set or sets of electrodes 571 used for resistivity measurement are preferably positioned on the blood-container wall such that their geometry remains largely unchanged (FIG. 7). For that, the electrodes 571 should be placed close to each other and they should have small dimensions for their shape remains essentially unchanged when the wall of the flexible blood-container changes shape. In addition, in the region where the electrodes 571 are placed, the wall of the blood-container 120 may be reinforced by increasing its thickness. The shape of the resistivity measuring electrodes 571 may be curved rectangular, or one electrode circular in the middle of an annular electrode, or other shapes suitable for the measurement.

Those skilled in the art will understand that, because the blood-container walls do not have a planar shape, the electrodes and other elements conventionally described as for a plane may have shapes corresponding to the specific three-dimensional shape of the blood-container 120. For example, if the bag is semi-spherical, the above-described electrodes 571 may be shaped as spherical polygons (rectangles) or as spherical cap (dome) instead of a circle, and as spherical segment instead a planar ring. FIGS. 7 and 10 show alternative shapes of the blood-container 120 in non-limiting embodiments.

Also, those skilled in the art will understand that several such pairs of electrodes 571 may be used and the results obtained can further be averaged for improving the accuracy of the overall measurement of the fluid resistivity (a higher number of pairs of electrodes can result in higher measurement accuracy), or for determining the local resistivity when the fluid is not homogeneous, as it may happen when blood and purulent fluids mix.

The fluid-measurement-system 550 may further include one or more of the following sensors: temperature sensors, ultrasound velocity sensors, colorimetric sensors, pH and electrochemical sensors. Fluid resistivity does not provide enough information about the composition of the fluid (e.g., percent of blood in fluid), as blood resistivity varies with temperature and other variables. Information provided by additional measurements/sensors (e.g. the sensors listed in this paragraph) enables the system to better estimate the composition of the fluid. For example, measurements by ultrasound velocity sensors are likely to provide useful information about the fluid composition since sound velocity is sensitive to the fluid composition. Colorimetric sensors are likely to provide composition information since they can easily distinguish between the red blood and the yellow purulent fluid (Szolga and Mudure, 2020), (Saetchnikov et al., 2010), (Trinder & Harper, 1962), (Dijkhuizen et al., 1977). PH and electrochemical sensors are likely to provide composition information since they respond to specific compounds (selective sensors to blood analytes, for example glucose, the level of which in the fluid can be compared with the level in pure blood).

A measurement cycle may include one or a combination of the following: low-frequency and high-frequency measurements of resistivity, electrical resistance measurements, impedance measurements, fluid density measurements, and temperature measurements. The measurements cycles may be repeated periodically thereby performing the monitoring of the fluid volume and fluid composition.

The electric resistivity and impedance measurement circuitry is of any known type. The switching between the low-frequency and the high-frequency resistivity measurements, the electrical resistance measurements, and impedance measurements may be performed automatically during each measurement cycle. Several implementations of electrodes and circuitry for performing such measurements are described for example in U.S. Pat. No. 11,009,383, Cumbie et al. (2021), which describes a set of electrodes and their placement for measuring the impedance of fluids in a printer, and in U.S. Pat. No. 10,966,668, Yi (2021), which uses several frequencies simultaneously and an elaborate computation system.

In some surgery circumstances, the quantity of bodily fluids overflowing from the body may vary significantly from case to case. When the fluid quantity is small, a significant part of it may remain on the drape and may not be measured by the fluid-measurement-system, thereby introducing errors in the blood loss estimates. In addition, when the quantity of fluid collected in the containers is small, the accuracy of fluid volume determination is lower. In such cases, it is preferable to have a system that has the capability of measuring both smaller and larger fluid quantities. Such a system is obtained by embodiments that combine the drape-blood-collectors embedded around the surgical place with blood-containers collecting fluids from the entire drape surface.

The fluid-measurement-system 550 may further include an optical system such as a camera configured to take pictures/images of the drape; to process the images via a processor; to recognize blood accumulations in the images (e.g., pools of blood and stains on the surface of the drape); and to estimate (e.g., in real time) the quantity of blood on the surface of the drape. The surface stained by the dried blood may be easily converted into quantities of blood unaccounted by the blood-containers. Combinations of the methods described above may be used to improve measurement accuracy of blood loss.

FIG. 8 shows an exemplary embodiment of a blood-management-system including a drape 110 which may have a saddle-shape, fluid-containers 120, and a set of gutters 600. The drape 110 and the gutters 600 have shapes configured to direct the fluids on the drape into the blood-containers 120. Some implementations of the blood-management-system including the drape necessitate that the drape essentially maintains its specified nonplanar surface over the entirety of its surface. In an exemplary embodiment, the flexible collecting drape 110 maintains, either by itself or with the help of a supporting structure, a shape essentially similar with a hyperbolic paraboloid surface (an essentially saddle-like shape), or a similar shape that directs the fluid flow toward one or several edges of the drape. The saddle shape (and similar ones, such as the monkey saddle shape) is particularly suitable, because it is a rolled surface, i.e., a surface such that, through any point of it passes at least one line fully contained in the surface. As a consequence of this particular property, various kinds of supporting structures/frames for the drape may be used which may include linear supporting elements. The supporting structures/frames enable the drape to essentially maintain the desired shape while remaining flexible. The hyperbolic paraboloid surface has two preferred directions of fluid flow over its surface, under gravity, which facilitates the collection of fluids, from a patient placed on an operation table, into blood-containers 120 suitably placed. In addition, the saddle (hyperbolic paraboloid) shape of the drape ensures that, even when the surgeon tilts by a moderate angle the surgery table, as can happen during surgeries, the flow directions on the surface remain essentially unchanged.

However, the hyperbolic paraboloid and other similar shapes, such as the ‘monkey-saddle’ shape allow fluid to flow on the entire lower edges of the saddle. To collect the fluid on these edges, the lower borders of the drape are provided with gutters 600 that direct any fluid they collect from the surface of the drape toward the blood-containers 120 (see FIGS. 8 and 9 for exemplary embodiments). A ‘monkey-saddle’ shape (Peckham 2011), (Weis stein, “Monkey Saddle”) of the drape can be chosen for drapes for gynecological surgery; further variations of shapes also suitable in some cases may be the starfish and octopus saddles or other variations (Peckham 2011), (Weis s tein, “Hankerchief Surface”).

In a nonlimiting embodiment, these types of drapes may use rigid supports/frames or inflatable supports/frames. For saddle-like drapes, a support/frame may have essentially the shape of the edges of the saddle whose surface must be assumed by the drape. This type of support/frame may be fixed to a surgical table.

FIG. 9 shows a non-limiting exemplary embodiment of a blood-management-system which may include a drape 110, fluid-containers 120, a set of gutters 600, a set of parabolic supports 700, and a set of linear supports 710. The supports may be inflatable supports. The 735 saddle-like drape is supported by the parabolic supports 700 (having parabolic shape) and linear supports 710. The linearity of supports 710 is allowed by the ruled surface property of the hyperbolic paraboloid. In another exemplary embodiment the blood-management-system includes a grid made of a few linear-only supports, either inflatable or solid, to support the saddle-like drape. Various solutions for deploying a hyperbolic paraboloid surface using pantographic structures with liner scissors or other structures based on linear elements have been described in the literature, for example see (Osmani et al., 2017).

In another nonlimiting embodiment, the blood collecting drape 110 may include a set of elastic strings or a set of rigid strings. The set of elastic strings may be attached/incorporated into the edges of the drape in the same way (or similar) that elastic strings are incorporated into the edges of elasticized bed linens (e.g., fitted sheets). The set of rigid strings may be attached/incorporated into the edges of the drape in the same way or similar to the draw strings attached into the upper edges of sport shorts/pants which enable the user to adjust the waist length of the shorts/pants. The strings enable the user to manipulate (e.g., shorten) the length of specific edges, thereby causing the edges to maintain an upward position. This way the edges are causing the fluids to flow along the edge (instead of perpendicular to the edge) and are directing the fluids towards the blood-container. The strings may be attached only to some of the edges of the drape, preferably the shorter edges.

The strings, when appropriately manipulated, shorten the length of the smaller edges of the drape and force them to preserve an upward position, in a similar way to the elasticized bed linen (sheets), while the longer sides of the drape can be lowered. The drape may further include gutters that are able of maintaining their shape (e.g., gutters may be rigid or semi-rigid), where the gutters are directing the fluids toward the collecting blood-containers 120. Preferably, the entire drape may be impermeable and/or fluid-repellant.

In another exemplary embodiment, the drape 110 may be an envelope-drape having a configuration like an envelope-type bed/comforter sheet with double walls. The envelope-drape may include two layers which are kept a small distance apart in the middle region around the surgical-site 103 (see e.g., FIG. 2(a)). The two layers may be kept apart by a low height distancing-piece (e.g., tenth of inch to one inch thick) so that the fluids around the surgical site and the surgery patch region enter between the two layers and naturally flow toward the edges of the drapes that hang from the surgery table. In this case, the fluid accumulates at the lower level between the two layers, from where it is collected in blood-containers 120. For this drape implementation, there is no need for gutters as the fluid is collected between the two layers of the envelope-like drape. Both layers should be fully impermeable and fluid-repellant. The blood-containers 120 are connected at the lowest regions of the drape (e.g., at the edge of the envelope-drape). The blood-containers 120 may be connected with the inside of the envelope-drape via one or more openings enabling the fluids to flow from the envelope into the blood-containers.

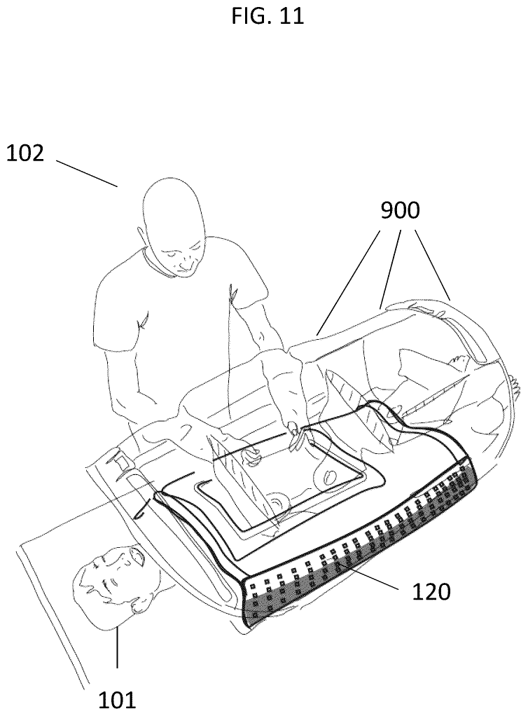

FIG. 11 shows an exemplary embodiment of the blood-management-system including an inflatable enclosure 900 such as the inflatable enclosures described in US patent application no. US20220192780A1 titled “ULTRAPORTABLE SYSTEM FOR INTRAOPERATIVE ISOLATIVE AND REGULATION OF SURGICAL SITE ENVIRONMENTS” and international patent application no PCT/US2021/058496 titled PORTABLE SYSTEM FOR ISOLATION AND REGULATION OF SURGICAL SITE ENVIRONMENTS which are incorporated herein. The operating surgical site may be included inside the enclosure 900. The enclosure 900 may include gloves and ports enabling operators to perform surgery on the surgical site inside the enclosure while isolating the surgical site from the outside environment. The drape 110 and the drape-blood-collectors 150 may form the basis of the enclosure 900 (placed on the patient). The enclosure prevents the blood and fluids from exiting into the environment outside the enclosure and directs the bodily fluids towards the collecting blood-containers 120. The blood-management-system and the inflatable enclosure may form a portable surgical system configured to be used for in the field applications (e.g., outdoors, in disaster relief areas and circumstances).

Those skilled in the art will understand that for all the implementations of drapes that do not maintain their shapes, the users may need to manipulate the drape such that the drape assumes a smooth surface without folds where fluids may accumulate and remain unaccounted for by the fluid-measurement-systems of the fluid containers 120. Also, the users may need to set-up and manipulate the blood-management-system such that the fluid containers 120 remain at a lower position than the drape and most of the blood flows from the drape into containers.

Those skilled in the art will understand that for the implementations of drapes that do not preserve their shapes, when the surface of the drape is very large, it may be difficult to maintain the entire surface without folds that may accumulate fluids. Such a situation may occur when the drape covers the inguinal region or the legs region of the patient. Further, those skilled in the art will understand that in such regions the drape may be provided with any combination of supplementary systems for locally collecting and measuring the bodily fluids.

### REFERENCES

[1]. Agnihotri, N., and Ajju A., “Transfusion associated circulatory overload.” Indian journal of critical care medicine: peer-reviewed, official publication of Indian Society of Critical Care Medicine vol. 18, 6 (2014): 396-8. doi:10.4103/0972-5229.133938.
[2]. L. Clifford et al., Risk Factors and Clinical Outcomes Associated with Perioperative Transfusion-associated Circulatory Overload. Anesthesiology 2017; 126:409-418 doi: https://doi.org/10.1097/ALN.0000000000001506.
[3]. J. A. Thornton, Estimation of Blood Loss During Surgery, 1963, Ann R Coll Surg Engl. 1963 September; 33(3): 164-174.
[4]. J. Desmond, A Method of Measuring Blood Loss during Transurethral Prostatic Surgery. J. Urology, Vol. 109, Issue 3, March 1973, Page: 453-456 https://doi.org/10.1016/50022-5347(17)60450-6.
[5]. M. H. Lee, B. T. Ingvertsen, J. Kirpensteijn, A. L. Jensen, and A. T. Kristensen, Quantification of Surgical Blood Loss. Veterinary Surgery 35:388-393, 2006.
[6]. R. S. Johar, R. P. Smith, Assessing Gravimetric Estimation of Intraoperative Blood Loss. Journal of Gynecologic Surgery, Vol. 9, No. 3, 2009 815 https://doi.org/10.1089/gyn.1993.9.151.
[7]. Alli, A., Hare, G. M. T., Kanga cloths to smartphones: how should we measure blood loss in the operating room? Can J Anesth/J Can Anesth 68, 175-179 (2021). https://doi.org/10.1007/s12630-020-01858-3.
[8]. Patent Application US20030060831A1 (Bonutti, 2003)
[9]. U.S. Pat. No. 11,160,602 by Shelton et al., 2021.
[10]. U.S. Pat. No. 5,167,613, (Karami and Vitaris, 1992);
[11]. U.S. Pat. No. 5,465,735 (Patel, 1995);
[12]. U.S. Pat. No. 6,077,526 (Scully and McCabe, 2000);
[13]. S. C. B. Mannsfeld et al., Highly sensitive flexible pressure sensors with microstructured rubber dielectric layers. Nature Materials, Vol 9, October 2010, pp. 859-864, www.nature.com/naturematerials.
[14]. Y. Kim, H. Yang, J. H. Oh, Simple fabrication of highly sensitive capacitive pressure sensors using a porous dielectric layer with cone-shaped patterns, Materials & Design, Vol. 197, 2021, 109203, https://doi.org/10.1016/j.matdes.2020.109203.
[15]. Y. Xiong et al., A flexible, ultra-highly sensitive and stable capacitive pressure sensor with convex microarrays for motion and health monitoring, Nano Energy, Vol. 70, 2020, 104436, https://doi.org/10.1016/j.nanoen.2019.104436.
[16]. Bouden et al., 2016
[17]. S. Luo et al., Tunable-Sensitivity flexible pressure sensor based on graphene transparent electrode, Solid-State Electronics, Vol. 145, 2018, Pages 29-33, https://doi.org/10.1016/j.sse.2018.04.003.
[18]. Mohapatra, S. N., Hill, D. W. The changes in blood resistivity with haematocrit and temperature. Europ. J. Intensive Care Med vol. 1, 153-162 (1975). https://doi.org/10.1007/BF00624433.
[19]. F. G. Hirsch, E. C. Texter, et al., The Electrical Conductivity of Blood: I. Relationship to Erythrocyte Concentration, Blood, Vol. 5, Issue 11, 1950, Pages 1017-1035, https://doi.org/10.1182/blood.V5.11.1017.1017.
[20]. H. P. Schwan, and C. F. Kay, Specific Resistance of Body Tissues. Circulation Research. Vol. IV, November 1956, p. 664-670.
[21]. Kellum J A., Determinants of blood pH in health and disease. Crit Care. 2000; 4(1):6-14. doi: 10.1186/cc644. Epub 2000 Jan. 24. PMID: 11094491; PMCID: PMC 137247.
[22]. L. A. Szolga and F. Mudure, “Blood (RBC/HGB) Analysis Using Colorimetry,” 2020 International Conference on e-Health and Bioengineering (EHB), 2020, pp. 1-4, doi: 10.1109/EHB50910.2020.9280195.
[23]. U.S. Pat. No. 10,088,454
[24]. Wong, W. S. and Salleo, A., Eds., “Flexible Electronics —Materials and Applications”. Springer Verlag 2009. doi:10.1007/978-0-387-74363-9.
[25]. V. A. Saetchnikov, E. A. Tcheriayskaia and G. Schweiger, “Point of care fiber optical sensor for non-invasive multi parameter monitoring of blood and human tissue biochemistry,” 2010 International Conference on Advanced Optoelectronics and Lasers, 2010, pp. 242-243, doi: 10.1109/CAOL.2010.5634197.
[26]. Trinder P, Harper F., A colorimetric method for the determination of carboxyhaemoglobin over a wide range of concentrations. J Clin Pathol. 1962 January; 15(1):82-4. doi: 10.1136/jcp.15.1.82.
[27]. Dijkhuizen et al., 1977
[28]. U.S. Pat. No. 11,009,383
[29]. U.S. Pat. No. 11,009,383, Cumbie et al., 2021
[30]. U.S. Pat. No. 10,966,668
[31]. Peckham, S. (2011). Monkey, Starfish and Octopus Saddles. Conf. on Geomorphometry, May 2011, Proceedings of the International Society for Geomorphometry At: Redlands, CA.
[32]. Weisstein, E. W. “Monkey Saddle.” From MathWorld—A Wolfram Web Resource. https://mathworld.wolfram.com/MonkeySaddle.html.
[33]. Weisstein, E. W. “Handkerchief Surface.” From MathWorld—A Wolfram Web Resource. https://mathworld.wolfram.com/HandkerchiefSurface.html.
[34]. A. Osmani, M. R. Matini, Y. Shahbazi, H. Golabi, Hyperbolic Paraboloid (Hp) Pantographic Structure with Liner Scissors, In Architecture Civil Engineering Environment, No. 4/2017, p. 89.
[35]. US patent application no. US20220192780A1.
[36]. International patent application no PCT/US2021/058496.

What is claimed is:

1. A blood-management-system for collecting blood from a surgical-site of a patient and for measuring the patient's blood-loss, the blood-management-system comprising:
a drape configured to be disposed on the patient, the drape comprising:
a surgical-site-layer configured to be disposed on a surgical-site of the patient and to provide an operator with access to the surgical-site;
an adhesive layer configured to form a seal between the drape and a region of the patient surrounding the surgical site;
a drape-collector disposed on the drape, in the proximity of the surgical-site, and adjacent with the surgical-site-layer, wherein the drape-collector is configured to collect blood spilled on the surgical-site-layer, to store blood, and to evaluate the quantity of blood collected in the drape-collector;
a blood-container configured to receive fluids from the drape, to store the fluids, and to measure the volume or the quantity of the fluids in the blood-container;
a computing system configured to receive information from the drape-collector and the blood-container and to evaluate the amount of blood lost by the patient.

2. The blood-management-system of claim 1, wherein the drape-collector comprises a fluids enclosure configured to collect and store fluids from the surgical-site-sheet.

3. The blood-management-system of claim 2, wherein the fluids-enclosure comprises an impermeable-upper-sheet, an impermeable-lower-sheet, and a permeable-sheet;
wherein the permeable-sheet is disposed over a surgical-site-edge of the drape-collector and is configured to allow fluids from the surgical-site-layer to enter the fluids-enclosure;
wherein the impermeable-upper-sheet is disposed on an outer-edge of the drape-collector and is configured to prevent liquids in the fluids-enclosure from spilling on the drape.

4. The blood-management-system of claim 3, wherein the drape-collector comprises one or more fluid-retention-elements disposed inside the fluids-enclosure, wherein the fluid-retention-elements are configured to absorb blood and fluids and to store blood and fluids in the fluids-enclosure.

5. The blood-management-system of claim 4, wherein the drape-collector comprises one or more pressure-sensors configured to measure the pressure generated by the fluids accumulated in the fluids-enclosure, wherein the pressure-sensors form one or more sensor arrays.

6. The blood-management-system of claim 5, wherein the pressure-sensors comprise a set of bottom-sensors disposed on the bottom of the fluids-enclosure and under the fluid-retention-elements.

7. The blood-management-system of claim 6, wherein the pressure-sensors comprise a set of side-sensors disposed on the side of the fluid-retention-elements and configured to measure pressure parallel to the drape.

8. The blood-management-system of claim 6, further comprising a signal-reading-system configured to receive the signals from the pressure-sensors and to send the signals to the computing system; wherein the computing-system is configured to evaluate the quantity of fluids accumulated in the fluids-enclosure based on the signals received from the pressure-sensors.

9. The blood-management-system of claim 3, wherein the fluids-enclosure comprises one or more tube drains configured to drain fluids from the drape-collector into an outside fluid container.

10. The blood-management-system of claim 5, wherein the drape-collector is formed as an essentially flexible layer contiguous with and incorporated into the drape; and wherein the sensors-arrays and the signal-reading-system are implemented via a flexible electronics technology.

11. The blood-management-system of claim 5, wherein the blood-container comprises:
a flexible-wall configured to adapt its shape to the surroundings;
a fluid-measurement-system configured to determine the volume of the fluid in the blood-container, the fluid-measurement-system comprising:
a plurality of electrodes disposed on the interior side of the flexible-wall and forming an electrodes-array;
a control-circuit configured to perform one or more of the following: control voltages on the electrodes; measure voltages on the electrodes; control currents through the electrodes; and measure currents through the electrodes.

12. The blood-management-system of claim 11,
wherein the computing-system is configured to control the electrical input on the electrodes and to receive electrical signals from the electrodes;
wherein the computing-system is configured to implement, via the electrodes, impedance-tomographic-measurements on the fluids in the blood-container and to evaluate the volume of the fluids in the blood-container based on the impedance-tomographic-measurements.

13. The blood-management-system of claim 12, wherein the computing-system is configured to implement, via the electrodes, electrical resistance tomographic measurements on the fluids in the blood-container and to evaluate the volume of the fluids in the blood-container based on the electrical-resistance-tomographic-measurements.

14. The blood-management-system of claim 13, further comprising a resistivity-measuring-system configured to measure the resistivity of the fluids in the blood-container, the resistivity-measuring-system comprising: two or more resistivity-measuring-electrodes disposed close to the bottom of the blood-container.

15. The blood-management-system of claim 12, wherein the fluid-measurement-system is configured to perform on the fluids in the blood-container one or more of the following: impedance tomographic measurements; resistance tomographic measurements; fluid resistivity measurements; fluid density measurements; colorimetric measurements; temperature measurements; pH measurements; and ultrasound measurements.

16. The blood-management-system of claim 15, wherein the fluids-measurement-system is further configured to evaluate the composition of the fluids in the blood-container or the amount of blood in the blood-container based on at least two of the following measurements: impedance tomographic measurements; resistance tomographic measurements; fluid resistivity measurements; fluid density measurements; colorimetric measurements; temperature measurements; pH measurements; and ultrasound measurements.

17. The blood-management-system of claim 11, wherein the drape is configured to collect blood and other fluids coming from the surgical-site and the patient and to direct the blood and the fluids into the blood-container.

18. The blood-management-system of claim 11, wherein the drape comprises one or more of the following: one or more raised edges; one or more gutters disposed at the edges; one or more rigid supports or frames; one or more inflatable supports or frames; and one or more rigid or elastic strings incorporated into the edges of the drape.

19. The blood-management-system of claim 17, wherein blood-container is a pocket blood-container disposed at the edge of the drape and at an elevation which is substantially lower than the drape.

20. The blood-management-system of claim 19, wherein the blood-container comprises one or more rigid or semi-rigid ribs configured to provide structure to the flexible walls of the blood-container.
